(19) United States
(12) Patent Application Publication
(10) Pub. No.: US 2025/0378910 A1
(43) Pub. Date: Dec. 11, 2025

(54) SYSTEMS AND METHODS TO IDENTIFY MUTATION AND PHENOTYPE ASSOCIATION

(71) Applicant: The Board of Regents of the University of Texas System, Austin, TX (US)

(72) Inventors: Darui Xu, Dallas, TX (US); Chun Hui Bu, Dallas, TX (US); Stephen Arthur Lyon, Dallas, TX (US); Yang Xie, Dallas, TX (US); Tao Wang, Dallas, TX (US); Xiaowei Zhan, Dallas, TX (US); Bruce Beutler, Dallas, TX (US)

(21) Appl. No.: 18/879,663

(22) PCT Filed: Jun. 21, 2023
(86) PCT No.: PCT/US2023/068787

§ 371 Date: Dec. 27, 2024

Related U.S. Application Data
(60) Provisional application No. 63/357,803, filed on Jul. 1, 2022.

(51) Int. Cl. G16B 20/40; G16B 40/20

(52) U.S. Cl. CPC G16B 20/40; G16B 40/20

(57) ABSTRACT

Aspects of the present inventive concept generally relate to systems and methods for mutation processing, and more specifically, for identifying associations between phenotypes and mutations. One example method generally includes receiving one or more input features including phenotype data and mutation data, generating, via a machine learning model, a candidate explorer (CE) score indicating a probability of association between a phenotype and a mutation based on the one or more input features, and outputting an indication of the association between the phenotype and the mutation based on the CE score.

### CROSS-REFERENCE TO RELATED APPLICATION

This application claims priority to U.S. Provisional Patent Application No. 63/357,803, filed Jul. 1, 2022 and titled “SYSTEMS AND METHODS TO IDENTIFY MUTATION AND PHENOTYPE ASSOCIATION,” the entirety of which is incorporated by reference herein.

### ACKNOWLEDGEMENT OF GOVERNMENT SUPPORT

This invention was made with government support under Grant Nos. A1125581 and A1100627 awarded by the National Institutes of Health. The government has certain rights in this invention.

### BACKGROUND

### 1. Technical Field

Aspects of the present inventive concept generally relate to systems and methods for mutation processing, and more specifically, for identifying associations between phenotypes and mutations.

### 2. Discussion of Related Art

A phenotype refers to a set of observable characteristics resulting from the interaction of a genotype with the environment. In some cases, a gene mutation may be causative for a phenotype. A mutation generally refers to a change in a deoxyribonucleic acid (DNA) sequence. Mutations can result from DNA copying made during cell division, ionizing radiation, mutagens, or infection by viruses.

### SUMMARY

Certain aspects of the disclosed technology can provide a method for mutation processing. The method can generally include receiving one or more input features including phenotype data and mutation data, generating, via a machine learning model, a candidate explorer (CE) score indicating a probability of association between a phenotype and a mutation based on the one or more input features, and outputting an indication of the association between the phenotype and the mutation based on the CE score.

In some examples, the indication of the association includes a candidate status for the association based on the CE score and an algorithmic score indicating a likelihood that the mutation is causative. The mutation data can include a damage score indicating a likelihood that a protein associated with the mutation is functionally impaired. Moreover, the method can include generating the damage score via another machine learning model trained using known deleterious and neutral mutations. Also, the one or more input features can further include an essentiality score indicating a likelihood of lethality prior to weaning age in mice homozygous for a robust knockout allele of a gene associated with the mutation. Additionally or alternatively, the method can include generating the essentiality score via another machine learning model trained using genes that are known to be non-essential for survival and genes that are known to be essential for survival. The one or more input features can further includes a feature associated with an algorithmic score indicating a likelihood that the mutation is causative.

Furthermore, in some instances, the one or more input features can further include linkage data generated using automated meiotic mapping (AMM). The method can also include, when two or more mutations are cosegregated, determining which of the two or more mutations is a more robust causation candidate for the phenotype by omitting instances of shared zygosity for the two or more mutations, wherein the CE score is generated based on the determination. Additionally, the one or more input features includes at least one of: a number of phenotypes with an algorithmic score for the mutation that meets a threshold, the algorithmic score indicating a likelihood that the mutation is causative; an average number of AMM operations resulting in a p-value that meets a threshold for each allele of a gene associated with the mutation; the algorithmic score for the mutation or phenotype; a number of AMM operations resulting in a p-value that meets a threshold for the gene associated with the mutation; a damage score for the mutation, the damage score indicating a likelihood that a protein associated with the mutation is functionally impaired; a number of pedigrees in a superpedigree associated with the gene and whether a p-value resultant from AMM operation for the superpedigree meets a threshold; a number of phenotypes with a p-value for the superpedigree that meets a threshold; a number of pedigrees contributing to a p-value for the superpedigree that meets a threshold; a number of pedigrees in the superpedigree; a percentage of fluorescence activated cell sorting (FACS) screens with a p-value that meets a threshold for the mutation; a minimum of the p-value from the AMM operations; a percentage of variant allele (VAR) mice with screen results that overlap with those of B6 mice; whether AMM operations results for the superpedigree meets a threshold for null and missense alleles; whether AMM operations results for the superpedigree meets a threshold for null alleles; a percentage of VAR mice with screen results that overlap with those of reference allele (REF) mice; a difference between results of AMM operations for heterozygous (HET) and VAR mice; a number of female REF mice used for the AMM operations; a percentage of body weight screens with a p-value that meets a threshold for the mutation; a number of female HET mice used for the AMM operations; and/or a difference between results of AMM operations for REF and VAR mice.

Additionally, certain aspects of the disclosed technology can provide an apparatus for mutation processing. The apparatus can generally include: a memory; and one or more processors coupled to the memory and configured to: receive one or more input features including phenotype data and mutation data, generate, via a machine learning model, a CE score indicating a probability of association between a phenotype and a mutation based on the one or more input features, and output an indication of the association between the phenotype and the mutation based on the CE score.

In some examples, the indication of the association includes a candidate status for the association based on the CE score and an algorithmic score indicating a likelihood that the mutation is causative. The mutation data can also include a damage score indicating a likelihood that a protein associated with the mutation is functionally impaired. Furthermore, the one or more processors can be further configured to generate the damage score via another machine learning model trained using known deleterious and neutral mutations. Also, the one or more input features can further include an essentiality score indicating a likelihood of lethality prior to weaning age in mice homozygous for a robust knockout allele of a gene associated with the mutation.

In some instances, the one or more processors can be further configured to generate the essentiality score via another machine learning model trained using genes that are known to be non-essential for survival and genes that are known to be essential for survival. The one or more input features can also include a feature associated with an algorithmic score indicating a likelihood that the mutation is causative. Additionally, the one or more input features can include linkage data generated using automated meiotic mapping (AMM). Furthermore, when two or more mutations are cosegregated, the one or more processors can be further configured to determine which of the two or more mutations is a more robust causation candidate for the phenotype by omitting instances of shared zygosity for the two or more mutations, wherein the one or more processors can be configured to generate the CE score based on the determination.

Certain aspects of the disclosed technology provide a non-transitory, computer-readable medium storing instructions that, when executed by one or more processors, cause the one or more processors to: receive one or more input features including phenotype data and mutation data, generate, via a machine learning model, a CE score indicating a probability of association between a phenotype and a mutation based on the one or more input features, and output an indication of the association between the phenotype and the mutation based on the CE score.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 illustrates an example computing device, in accordance with certain aspects of the present inventive concept.

FIG. 2 is a diagram illustrating input and output features of a candidate explorer (CE) system, in accordance with certain aspects of the present inventive concept.

FIG. 3 is graph illustrating a polynomial regression analysis of a CE score and average percentage of verified mutation-phenotype associations, in accordance with certain aspects of the present inventive concept.

FIG. 4 is a graph illustrating a receiver operating characteristic (ROC) curve for a CE score, in accordance with certain aspects of the present inventive concept.

FIG. 5A is a table showing CE performance for flow cytometry phenotypes, in accordance with certain aspects of the present inventive concept.

FIG. 5B is a table showing CE performing in scoring colocalizing mutations, in accordance with certain aspects of the present disclosure.

FIG. 6 is a table showing examples of input features to a machine learning model of the CE system, in accordance with certain aspects of the present inventive concept.

FIG. 7 is a table showing rules for algorithmic score determination, in accordance with certain aspects of the present inventive concept.

FIG. 8 is a graph illustrating an ROC curve for the algorithmic score, in accordance with certain aspects of the present inventive concept.

FIG. 9 is a table showing flow cytometry screening parameters, in accordance with certain aspects of the present inventive concept.

FIG. 10A is a graph showing the number of good/excellent phenotype associations plotted versus gene count, in accordance with certain aspects of the present inventive concept.

FIG. 10B shows the number of good/excellent gene associations plotted versus flow cytometry parameters, in accordance with certain aspects of the present inventive concept.

FIG. 10C shows the number and percentage of essential and non-essential genes, in accordance with certain aspects of the present inventive concept.

FIG. 11 is a flow diagram illustrating example operations for mutation processing, in accordance with certain aspects of the present inventive concept.

It will be apparent to one skilled in the art after review of the entirety disclosed that the steps illustrated in the figures listed above may be performed in other than the recited order, and that one or more steps illustrated in these figures may be optional.

### DETAILED DESCRIPTION

Certain aspects of the present inventive concept are directed to methods and systems for using a machine-learning algorithm to identify chemically induced mutations that are causative of screened phenotypes. For example, a candidate explorer (CE) system may determine the probability that a mutation will be verified as causative for a phenotype if the gene is independently targeted for knockout or recreation of the mutation. The CE system (also referred to in short as “CE”) uses a number of parameters (e.g., 67 parameters) from mapping data, including gene, mutation, genotype, allelism, and phenotype information, to determine a CE Score and verification probability.

Forward genetic studies use meiotic mapping to adduce evidence that a particular mutation, normally induced by a germline mutagen, is causative of a particular phenotype. Particularly in small pedigrees, cosegregation of multiple mutations, occasional unawareness of mutations, and paucity of homozygotes may lead to erroneous declarations of cause and effect. Certain aspects of the present inventive concept provide systems to improve the identification of mutations causing immune phenotypes which may be identified in mice. The CE system may use machine learning to integrate features of genetic mapping data into a single numeric score, mathematically convertible to the probability of verification of any putative mutation-phenotype association.

The CE system may be used to evaluate putative mutation-phenotype associations arising from screening damaging mutations in (e.g., about 55% of) mouse genes for effects on flow cytometry measurements of immune cells in the blood. The CE system may identify more than half of genes within which mutations can be causative of flow cytometric phenovariation in Mus musculus (e.g., house mouse). The majority of these genes may not be previously known to support immune function or homeostasis. Mouse geneticists may use CE data to identify causative mutations within quantitative trait loci. A quantitative trait locus is a region of DNA which is associated with a particular phenotypic trait. Clinical geneticists may use CE to help connect causative variants with rare heritable diseases of immunity, even in the absence of linkage information. CE displays integrated mutation, phenotype, and linkage data.

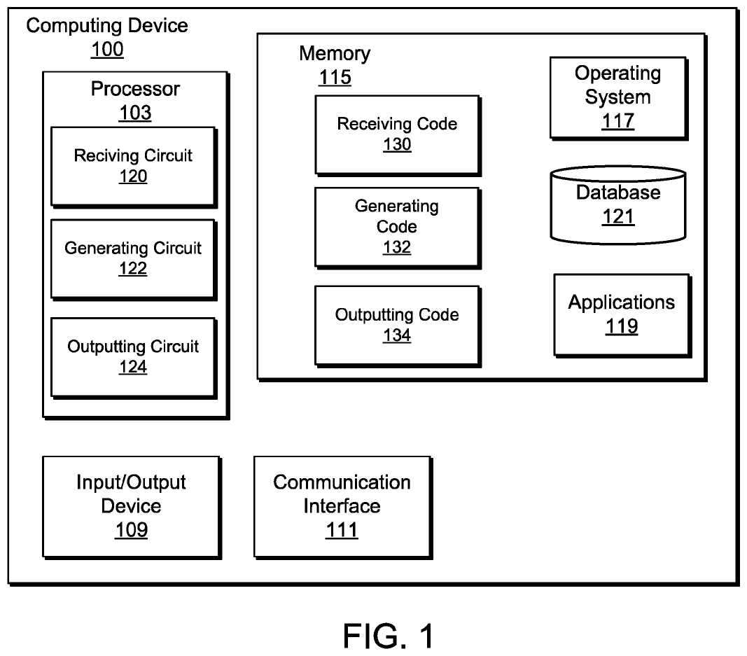

FIG. 1 illustrates an example computing device 100, in accordance with certain aspects of the present inventive concept. The computing device 100 can include a processor 103 for controlling overall operation of the computing device 100 and its associated components, including input/output device 109, communication interface 111, and/or memory 115. A data bus can interconnect processor(s) 103, memory 115, I/O device 109, and/or communication interface 111.

Input/output (I/O) device 109 can include a microphone, keypad, touch screen, and/or stylus through which a user of the computing device 100 can provide input and can also include one or more of a speaker for providing audio output and a video display device for providing textual, audiovisual, and/or graphical output. Software can be stored within memory 115 to provide instructions to processor 103 allowing computing device 100 to perform various actions. For example, memory 115 can store software used by the computing device 100, such as an operating system 117, application programs 119, and/or an associated internal database 121. The various hardware memory units in memory 115 can include volatile and nonvolatile, removable, and non-removable media implemented in any method or technology for storage of information such as computer-readable instructions, data structures, program modules or other data. Memory 115 can include one or more physical persistent memory devices and/or one or more non-persistent memory devices. Memory 115 can include, but is not limited to, random access memory (RAM), read only memory (ROM), electronically erasable programmable read only memory (EEPROM), flash memory or other memory technology, CD-ROM, digital versatile disks (DVD) or other optical disk storage, magnetic cassettes, magnetic tape, magnetic disk storage or other magnetic storage devices, or any other medium that can be used to store the desired information and that can be accessed by processor 103.

Communication interface 111 can include one or more transceivers, digital signal processors, and/or additional circuitry and software for communicating via any network, wired or wireless, using any protocol as described herein. Processor 103 can include a single central processing unit (CPU), which can be a single-core or multi-core processor (e.g., dual-core, quad-core, etc.), or can include multiple CPUs. Processor(s) 103 and associated components can allow the computing device 100 to execute a series of computer-readable instructions to perform some or all of the processes described herein. Although not shown in FIG. 1, various elements within memory 115 or other components in computing device 100, can include one or more caches, for example, CPU caches used by the processor 103, page caches used by the operating system 117, disk caches of a hard drive, and/or database caches used to cache content from database 121. For implementations including a CPU cache, the CPU cache can be used by one or more processors 103 to reduce memory latency and access time. A processor 103 can retrieve data from or write data to the CPU cache rather than reading/writing to memory 115, which can improve the speed of these operations. In some examples, a database cache can be created in which certain data from a database 121 is cached in a separate smaller database in a memory separate from the database, such as in RAM or on a separate computing device. For instance, in a multi-tiered application, a database cache on an application server can reduce data retrieval and data manipulation time by not needing to communicate over a network with a back-end database server. These types of caches and others can be included in various implementations and can provide potential advantages in certain implementations of software deployment systems, such as faster response times and less dependence on network conditions when transmitting and receiving data.

Forward genetics begins with a phenotype, often induced by a random germline mutagen, and ends with the discovery of a causative mutation. Certain aspects provide techniques for rapid identification of causative mutations in mice carrying N-ethyl-N-nitrosourea (ENU)-induced germline mutations. Certain aspects provide techniques involving mutagenizing a male inbred strain (e.g., C57BL/6J) of mice (G0) mice and breeding them on the C57BL/6J background to create first-generation (G1) male pedigree founders, second-generation (G2) daughters, and third-generation (G3) mice of both sexes. The exomes of all G1 founders of pedigrees may be sequenced to achieve greater than 99% 10× coverage over the targeted exome. Identified variants (e.g., with respect to the C57BL/6J reference genome) are genotyped in G2 and G3 mice in advance of phenotypic screening. Using a variety of phenotypic screens, G3 mice may be then tested for phenovariance with respect to C57BL/6J mice or a control population of G3 mice. Demonstrating linkage between a mutant phenotype detected in screening and a particular mutation is accomplished by automated meiotic mapping (AMM) performed by a linkage analyzer algorithm (or program or software), which tests a null hypothesis for every mutation in the pedigree (e.g., “mutation A is unrelated to phenotypic performance in screen a”). In contrast, a mutation associated with the mutant phenotype at a frequency greater than predicted by chance alone is likely to confer the phenotype. Rejection of the null hypothesis with a p-value of less than or equal to 0.05, with Bonferroni correction for multiple comparisons, may be considered suggestive of causation. Verification by an independently generated allele may be used to confirm the association.

Experience with many thousands of mutation-phenotype associations identified by AMM and either verified or excluded by testing CRISPR/Cas9-targeted alleles, has shown that the p-value determined by AMM is not the sole indicator of causation. A p-value is a measure of the probability that an observed difference could have occurred just by random chance. The lower the p-value, the greater the statistical significance of the observed difference.

A mutation linked to a phenotype with a p-value less than 0.05 is sometimes not the causative mutation. Many other factors, such as the nature of the mutation (benign, damaging, null), the essentiality of the gene for survival prior to weaning, pedigree size, the number of homozygotes tested, the magnitude of phenotypic effect, data variance characteristics of the screen in question, the number of distinct phenotypes caused by the mutation, the presence or absence of cosegregating mutations, and the observation of other alleles with similar effects, influence the correct selection of an authentic causative mutation. The CE system described herein may estimate the likelihood of verification of any putative mutation-phenotype association implicated by AMM.

Changes in immune cell populations, specifically B cells, T cells, conventional and plasmacytoid dendritic cells (DC), macrophages, neutrophils, natural killer (NK) cells, and NK1.1⁺ T cells may be analyzed. Cell populations and subpopulations may be detected and measured by flow cytometric analysis of peripheral blood leukocytes from G3 mutant mice carrying ENU-induced mutations. In some cases, the CE system has been used to assess 87,795 mutation-phenotype associations (e.g., having P<0.05), from which the CE system has identified more than 1,270 genes with a high and defined probability of verifiable importance in leukocyte development or maintenance. Many of the genes were not previously known to be important in immune function.

The CE system may aid a researcher in predicting whether a mutation associated with a phenotype by AMM is a truly causative mutation. The CE system evaluates mutation-phenotype associations that pass specific basal filters for conventionally good candidates. Default filters of data that may be used include a p-value of less than 0.05 (Bonferroni corrected), ≥10 mice in the tested pedigree, and ≥2 homozygous reference mice screened; however, more stringent criteria can be set by a user. The core of CE system is a supervised machine-learning algorithm that outputs a numerical score (CE score), a categorical assessment (candidate status), and verification probability for each mutation-phenotype association based on input phenotype data (e.g., from screening), mutation data, gene data, and meiotic mapping data.

Referring to FIG. 1, the processor 103 and/or memory 115 may be used to implement the CE system. For example, the processor 103 may include circuit 120 for receiving one or more input features (e.g., receiving at least one of phenotype features, linkage data features, mutation features, gene features, or an algorithmic score). The processor 103 may also include circuit 122 for generating a CE score based on the one or more input features. For example, the circuit 122 may be a trained machine learning model. The machine learning model may be trained based on phenotypic assessment of mice carrying targeted null or replacement alleles of candidate genes. The processor 103 may also include circuit 124 for outputting an indication of an association between a phenotype and a mutation based on the CE score.

The memory 115 may be coupled to processor 103 and may store code which, when executed by the processor 103, performs the operations described herein. For example, the memory 115 may include code 130 for receiving one or more input features, code 132 for generating a CE score, or code 134 for outputting the indication of association.

As described, the CE system may include a machine learning model. The machine learning model may be trained using an objective function. For example, candidate solutions may be provided to the model and evaluated against training datasets. An error score (also referred to as a loss of the model) may be calculated by comparing the solution with the training dataset. The machine learning model may be trained to minimize the error score. For example, the machine learning model may be trained to implement a CE system, including the memory 115 and processor 103. The CE system may be trained based on a phenotypic assessment of mice carrying targeted null or replacement alleles of candidate genes. In predicting, performed four times per day because of the dynamic status of the database, CE uses all defined features of the original pedigree screening data to estimate the probability of candidate verification. CE may be used for querying mutation-phenotype associations identified in flow cytometry screens, as well as radiographic screens of bone (dual-energy X-ray absorptiometry (DEXA) scanning).

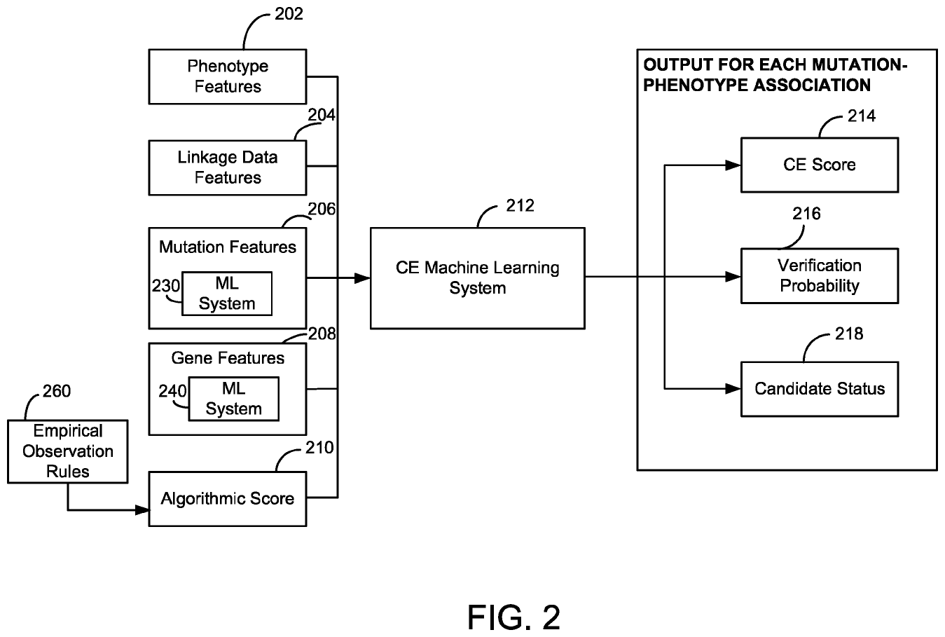

FIG. 2 is a diagram illustrating input and output features of the CE system, in accordance with certain aspects of the present inventive concept. The CE machine learning system 212 may be a supervised machine-learning algorithm that outputs a numerical score (e.g., CE score 214), a categorical assessment (e.g., candidate status 218), and verification probability 216 for each mutation-phenotype association based on various input features. The input features may include input phenotype data (e.g., phenotype features 202), mutation data (e.g., mutation features 206), gene data (e.g., gene features 208), meiotic mapping data (e.g., linkage data features 204), and an algorithmic score 210. The mutation features may include a damage score indicating a likelihood that a protein associated with a mutation is functionally impaired. The damage score may be generated using a ML system 230, which may be using known deleterious and neutral mutations, as described in more detail herein. The gene features 208 may include an essentiality score indicating a likelihood of lethality prior to weaning age in mice homozygous for a robust knockout allele of a gene associated with the mutation. The essentiality score may be generated using an ML system 240 which may be trained using genes that are known to be non-essential for survival and genes that are known to be essential for survival. The algorithmic score may be a score generated based on a set of rules 260 associated with empirical observations, as described in more detail herein. The meiotic mapping data may be generated using automated meiotic mapping (AMM) as described herein. The generated CE score 214 may be used to determine the verification probability 216. The CE score, along with the algorithmic score, may be used to generate the candidate status 218 (e.g., whether the mutation-phenotype association is an excellent, good, potential, or not good candidate).

As described herein, the CE system may be trained using a CE training set. The CE training set (e.g., used to train the machine learning model of the CE system 212) may contain verified (e.g., 1,903 verified) and excluded (e.g., 3,013 excluded) mutation-phenotype associations (4,916 assessments in all), based on germline retargeting of genes (e.g., 514 genes). Germline retargeting may be performed using CRISPR/Cas9 to generate knockout alleles of candidate genes in mice on a pure reference background (C57BL/6J or C57BL/6N). Alternatively, when evidence for homozygous lethality of null alleles exists (e.g., using an essentiality score as described herein) or the N-ethyl-N-nitrosourea (ENU) mutation is suspected to cause hypermorphic, neomorphic, or antimorphic effects, the original ENU allele may be recreated by CRISPR/Cas9 targeting (designated “replacement” allele). Mice carrying targeted germline knockout or replacement alleles may be expanded to form pedigrees containing mice homozygous for reference allele (REF), heterozygous (HET), and homozygous for the variant allele (VAR). Compound heterozygous mice with two or more variant alleles of a gene may be generated. Fresh pedigrees of mice carrying the CRISPR-targeted alleles may be subjected to the phenotypic screens in which the original ENU mutations scored as hits. In some aspects, CRISPR-targeted mutations may be considered verified according to criteria including (1) observation of the same phenotype with the same directionality of change as observed for the original ENU allele with a p-value better than 0.01, (2) observation of the same phenotype with the opposite directionality of change as observed for the original ENU allele with a p-value better than 0.001, or (3) de novo observation of a phenotype (e.g., not seen in the original screen) with a p-value better than 0.001.

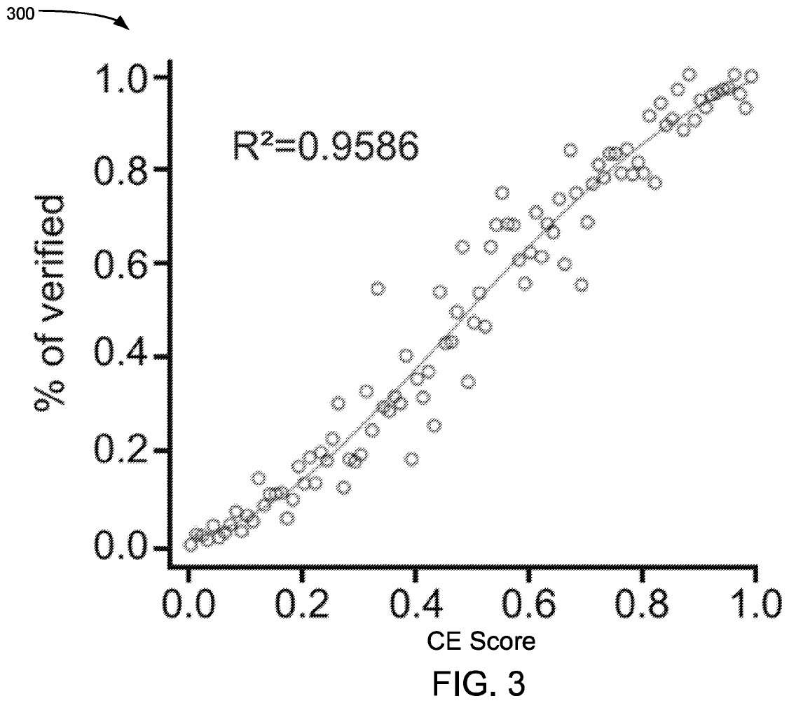

FIG. 3 is graph 300 illustrating a polynomial regression analysis of CE score and average percentage of verified mutation-phenotype associations, in accordance with certain aspects of the present inventive concept. Each data point represents a group of mutation-phenotype associations. The percentage of verified associations (e.g., on the y-axis of graph 300) is plotted versus a CE score range (e.g., on the x-axis of graph 300) in bins of 0.01 (e.g., 0.35 to 0.36, 0.37 to 0.38, and so forth), where n=4,916 mutation-phenotype associations and 514 CRISPR/Cas9-targeted genes. The CE score (e.g., ranging from 0 to 1) is a class probability related by a polynomial function to the actual probability of verification by CRISPR-targeted alleles, as determined by the regression analysis. In conjunction with the algorithmic score, it is used by the CE system to designate one of four possible candidate statuses for each mutation-phenotype association (excellent, good, potential, or not good). In some aspects, an excellent candidate corresponds to a CE score≥0.39 and algorithmic score≥−0.5, a good candidate corresponds to a CE score≥0.39 and −4.5≤algorithmic score<−0.5, a potential candidate corresponds to a CE score≥0.39 and algorithmic score<−4.5 or a CE score<0.39 and algorithmic score≥−0.5, and a not good candidate corresponds to a CE score<0.39 and algorithmic score<−0.5.

In some aspects, good or excellent candidates for CRISPR/Cas9 targeting and further study may be chosen. However, CE scores are not strictly proportional to the probability of verification as shown in FIG. 3, and some “good” or “excellent” candidates may fail to verify. Conversely, “potential” and “not good” candidates will sometimes verify as true positive associations. Authentic candidates may achieve strong CE scores as more alleles are obtained and tested (e.g., approaching saturation) and may therefore eventually be verified.

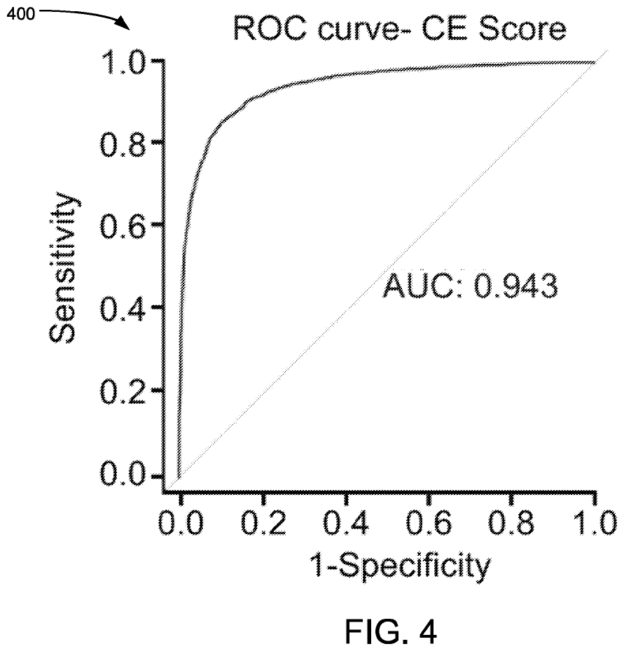

FIG. 4 is a graph 400 illustrating a receiver operating characteristic (ROC) curve for CE score, in accordance with certain aspects of the present inventive concept. The performance of the CE prediction model established using the training set may be assessed using the repeated 10-fold cross-validation method. The ROC curve has an area under the curve (AUC) of 0.943, where the cutoff may be set to 0.39, corresponding to the point with the minimum distance to the upper left corner of the ROC curve.

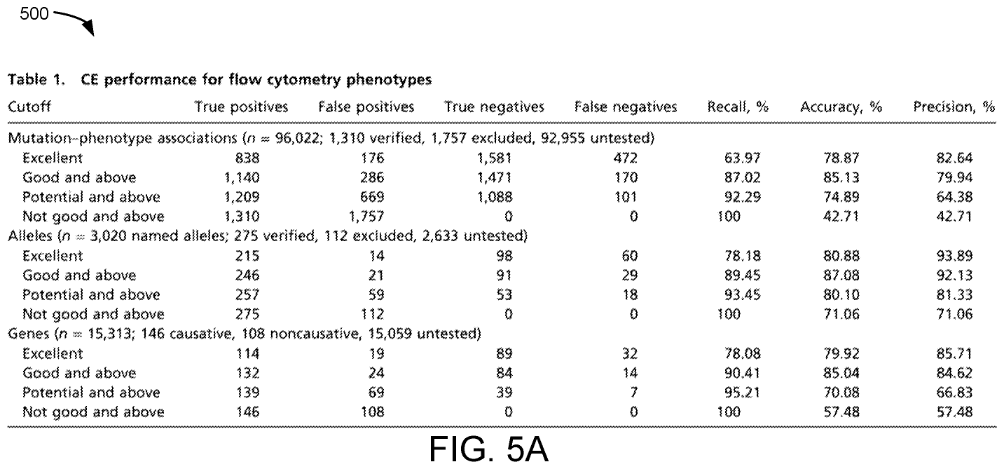

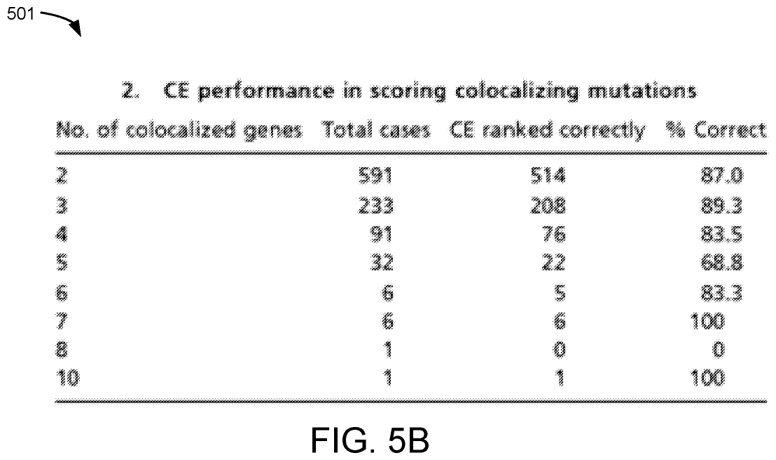

FIG. 5A is a table 500 showing CE performance for flow cytometry phenotypes, in accordance with certain aspects of the present inventive concept. As shown, CE ranking of good or better may correspond to about 80% precision (e.g., correctly calling a verified candidate “true,” a 20% false-discovery rate) and 87% recall (e.g., a true positive rate).

FIG. 5B is a table 501 showing CE performance in scoring colocalizing mutations, in accordance with certain aspects of the present inventive concept. The CE system may identify which mutation is causative when two or more mutations cosegregate (e.g., determined by a driven by software, as described herein). Among 961 such cases, CE may identify on average 76.5% of causative mutations as the top CE scorer, with generally better performance when fewer mutations cosegregated, as shown in FIG. 5B. As further training is performed, CE performance will continue to improve as the total volume of screening data increases (e.g., with an attendant increase in the number of genes with allelism and the overall density of allelic series).

Multiple alleles of a given gene may be subjected to a given phenotypic screen, resulting in several mutation-phenotype associations for the same gene and phenotype. Each mutation-phenotype association may be independently accorded an allele verification probability (AVP) estimate for the mutation in question, extrapolated from the polynomial regression analysis of CE score and the average percentage of verified mutation-phenotype associations (e.g., as shown in FIG. 3). In addition, the composite estimate that one or more mutations (e.g., N mutations) within a certain gene may be verified as the source of a certain phenotype (e.g., gene verification probability (GVP)) is given by:

$$G V P = 1 - ( 1 - {AVP}_{1} ) ( 1 - {AVP}_{2} ) ( 1 - {AVP}_{3} ) . . . ( 1 - {AVP}_{N} ) .$$

AVPs of alleles causing the same direction of phenotypic change in a given screen are included in the calculation.

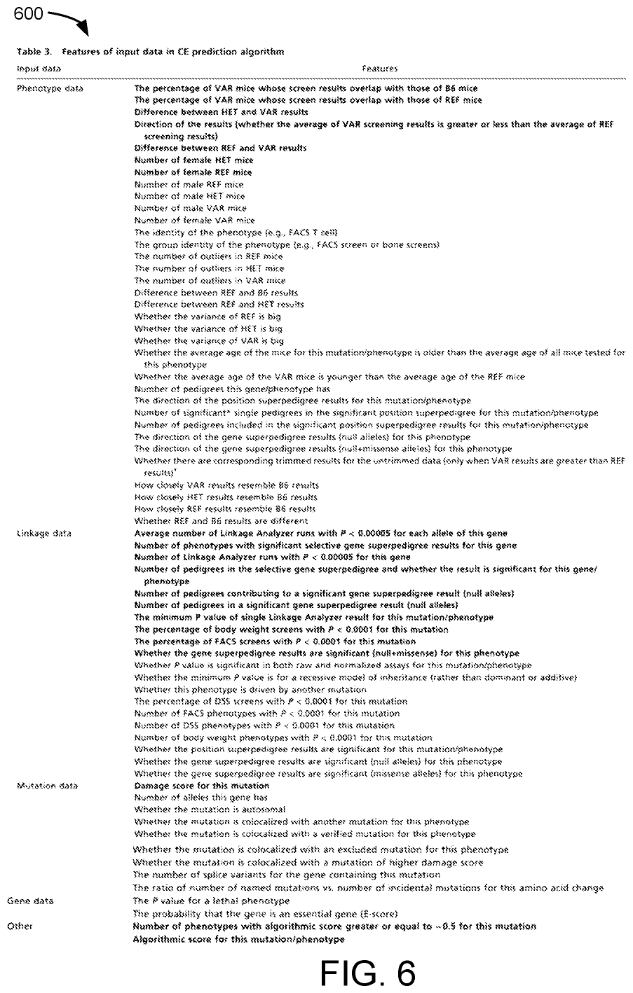

FIG. 6 is a table 600 of input features to a machine learning model of the CE system, in accordance with certain aspects of the present inventive concept. The CE prediction model may incorporate 67 features of input data, including thirty-four phenotype features (e.g., phenotype features 202 of FIG. 2), twenty linkage analysis features (e.g., linkage analysis features 204 of FIG. 2), nine mutation features (e.g., mutation features 206 of FIG. 2), two gene features (e.g., gene features 208 of FIG. 2), and two other features (e.g., algorithmic score 210 of FIG. 2).

Table 600 provides examples of input features. For example, the phenotype features may include at least one of the percentage of VAR mice whose screen results overlap with those of B6 mice, the percentage of VAR mice whose screen results overlap with those of REF mice, difference between HET and VAR results, direction of the results (whether the average of VAR screening results is greater or less than the average of REF screening results), difference between REF and VAR results, number of female HET mice, number of female REF mice, number of male REF mice, number of male HET mice, number of male VAR mice, number of female VAR mice, the identity of the phenotype (e.g., fluorescence-activated cell sorting (FACS) T cell), the group identity of the phenotype (e.g., FACS screen or bone screens), the number of outliers in REF mice, the number of outliers in HET mice, the number of outliers in VAR mice, difference between REF and B6 results, difference between REF and HET results, whether the variance of REF is big (e.g., is above a threshold), whether the variance of HET is big (e.g., is above a threshold), whether the variance of VAR is big (e.g., is above a threshold), whether the average age of the mice for this mutation/phenotype is older than the average age of all mice tested for this phenotype, whether the average age of the VAR mice is younger than the average age of the REF mice, number of pedigrees this gene/phenotype has, the direction of the position superpedigree results for this mutation/phenotype, number of significant single pedigrees in the significant position superpedigree for this mutation/phenotype (e.g., where significant pedigree refers to linkage analysis of a pedigree or superpedigree by AMM in which p-value<0.05 for a mutation-phenotype association), number of pedigrees included in the significant position superpedigree results for this mutation/phenotype, the direction of the gene superpedigree results (null alleles) for this phenotype, the direction of the gene superpedigree results (null+missense alleles) for this phenotype, whether there are corresponding trimmed results for the untrimmed data (e.g., only when VAR results are greater than REF results) where the trimmed results are raw data normalized for cell viability, how closely VAR results resemble B6 results, how closely HET results resemble B6 results, how closely REF results resemble B6 results, or whether REF and B6 results are different. Linkage features may include at least one of the average number of Linkage Analyzer runs with p-value<0.00005 for each allele of the gene, number of phenotypes with significant selective gene superpedigree results for this gene, number of Linkage Analyzer runs with p-value<0.00005 for this gene, number of pedigrees in the selective gene superpedigree and whether the result is significant for this gene/phenotype, number of pedigrees contributing to a significant gene superpedigree result (null alleles), number of pedigrees in a significant gene superpedigree result (null alleles), the minimum p-value of single Linkage Analyzer result for this mutation/phenotype, the percentage of body weight screens with p-value<0.0001 for this mutation, the percentage of FACS screens with p-value<0.0001 for this mutation, whether the gene superpedigree results are significant (null+missense) for this phenotype, whether p-value value is significant in both raw and normalized assays for this mutation/phenotype, whether the minimum p-value value is for a recessive model of inheritance (rather than dominant or additive), whether this phenotype is driven by another mutation, the percentage of DSS screens with p-value<0.0001 for this mutation, number of FACS phenotypes with p-value<0.0001 for this mutation, number of Dejerine-Sottas syndrome (DSS) phenotypes with p-value<0.0001 for this mutation, number of body weight phenotypes with p-value<0.0001 for this mutation, whether the position superpedigree results are significant for this mutation/phenotype, whether the gene superpedigree results are significant (null alleles) for this phenotype, or whether the gene superpedigree results are significant (missense alleles) for this phenotype.

The mutation features (e.g., mutation features 206) may include at least one of a damage score for the mutation, number of alleles the gene has, whether the mutation is autosomal, whether the mutation is colocalized with another mutation for this phenotype, whether the mutation is colocalized with a verified mutation for this phenotype, whether the mutation is colocalized with an excluded mutation for this phenotype, whether the mutation is colocalized with a mutation of higher damage score, the number of splice variants for the gene containing this mutation, or the ratio of number of named mutations vs. number of incidental mutations for this amino acid change. The gene features may include at least one of the p-value for a lethal phenotype or the probability that the gene is an essential gene (e.g., based on a calculated E-score as described herein). Other features (e.g., algorithmic score features) may include at least one of the number of phenotypes with an algorithmic score greater or equal to −0.5 for this mutation or an algorithmic score for this mutation/phenotype. While various features are shown in table 600, only a subset of the features may be used, such as the features shown in bold font.

In some aspects, the damage score and essentiality score (E-score) result from independent machine-learning programs. The rule-based algorithmic score results from the computational execution of a fixed algorithm.

The damage score (e.g., ranging from 0 to 1), a mutation feature, has important biological relevance. The damage score denotes the likelihood that a protein is functionally impaired and is determined by a machine-learning algorithm that integrates independent prediction scores (e.g., 37 scores) from the human database for Nonsynonymous Functional Prediction (dbNSFP) and the probability of protein damage to phenovariance caused by mouse mutations. A higher score suggests a mutation is more likely to be deleterious, and therefore more likely to be causative (although not always the case). The damage score prediction model may be implemented using a machine learning model trained on known deleterious mutations (e.g., 871 mutations) and known neutral mutations (e.g., 1,797 mutations). Mutations (e.g., 666 mutations) with known effects may be used to test the performance of the established model, which may yield an ROC curve with AUC of 0.852. A deleterious mutation refers to a genetic alteration that increases a susceptibility or predisposition to a certain disease or disorder. A neutral mutation refers to a mutation that is neither beneficial nor detrimental to the ability of an organism to survive and reproduce.

The E-score (e.g., ranging from 0 to 1) is a gene feature and denotes the likelihood of lethality prior to weaning age (e.g., 4 week postpartum) in mice homozygous for a robust knockout allele of a gene. The E-score is calculated using a machine-learning algorithm incorporating various independent features of genes, including gene conservation, protein-protein interaction network, expression stage, and viability/proliferative ability of human cell lines in which the gene is mutated. The machine learning model (e.g., also referred to as an E-score prediction model) for generating the E-score may be trained on lethal/viable mutations. The E-score prediction model may be trained at monthly intervals. The training dataset may include known non-essential genes (E-score=0) (e.g., 3,538 non-essential genes) and known essential genes (E-score=1) (e.g., 2,070 essential genes), determined based on annotations in a mouse genome Informatics (MGI) database and observed effects of CRISPR-targeted null mutations generated in C57BL/6J mice. The cutoff values may be set to greater than 0.5 for essential genes and less than 0.5 for non-essential genes, and are used to inform gene-targeting efforts, in which either a knockout allele or a replacement identical to the original ENU allele is created for verification of a phenotype. Genes (e.g., 1041 genes) with known effects on viability may be used to test the performance of the established model, which may yield an ROC curve with AUC of 0.894.

Assessments of mutation-phenotype associations may be made using a human-developed algorithm that outputs a points-based score called the algorithmic score (e.g., having a range from −13.5 to 3.5). The algorithmic score appears twice among important features contributing to the CE algorithm and provides an overall assessment of how likely the mutation is to be causative.

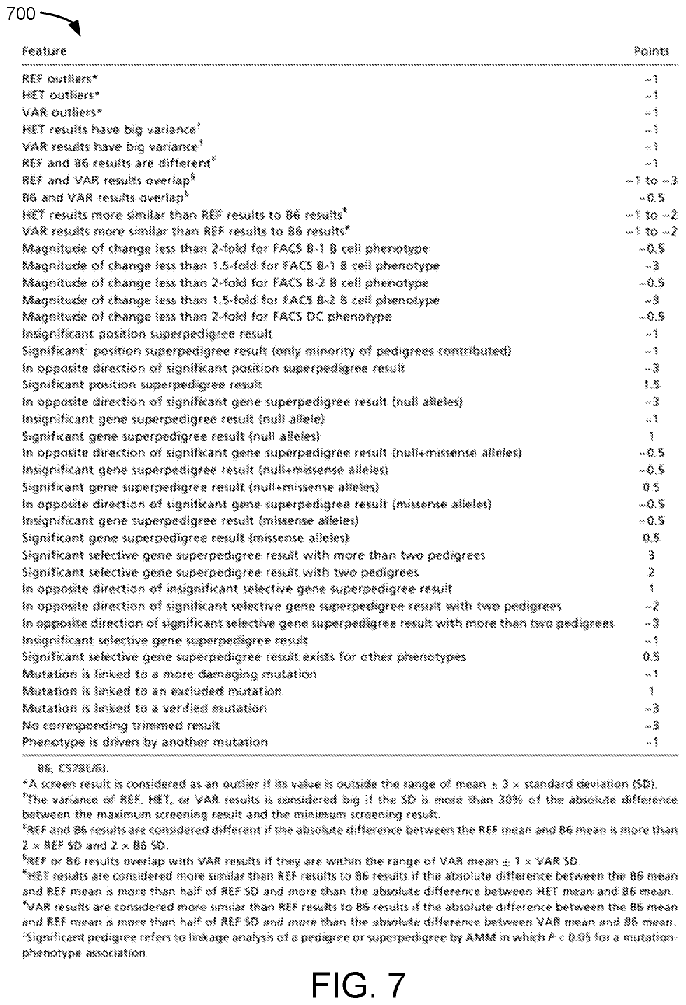

FIG. 7 is a table 700 showing rules for algorithmic score determination, in accordance with certain aspects of the present inventive concept. The algorithm includes a set of rules based on empirical observations. For each feature supporting or opposing the authenticity of a mutation-phenotype association, respectively, the algorithmic score is increased or decreased. The features used in the algorithmic score calculation are similar to those used in the CE machine-learning algorithm, but static (e.g., not influenced by exposure to new training data), and the performance of the rule-based algorithm by itself falls short of the performance of the CE prediction model. Each mutation-phenotype association starts with an algorithmic score of zero that is adjusted according to the rules described herein with respect to FIG. 7.

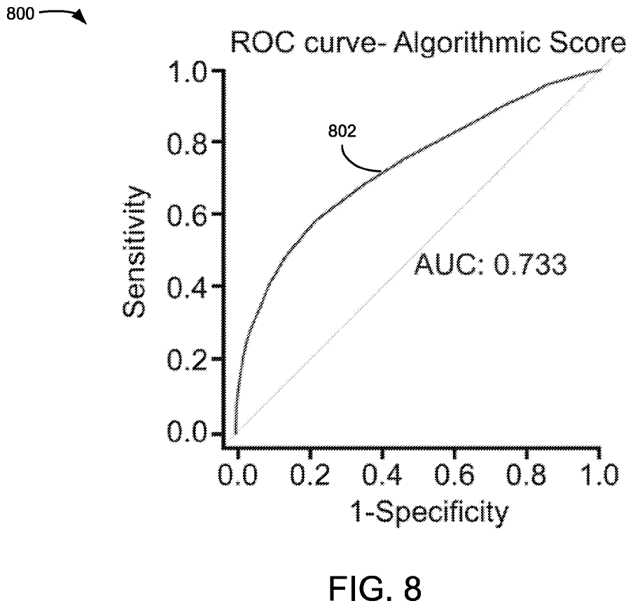

FIG. 8 is a graph 800 illustrating an ROC curve 802 for the algorithmic score. As shown, the AUC for the ROC curve 802 is 0.733 which is below the performance of the CE prediction model having an AUC of 0.943. Other input features (e.g., linkage data features 204) to the CE algorithm may be generated by an algorithm called a driven by algorithm, which evaluates linked and unlinked candidate mutations to determine the best candidate. A cluster of linked mutations sometimes fails to undergo meiotic separation; hence, more than one mutation may stand as a candidate for causation of a phenotype. On other occasions, as a matter of happenstance, homozygotes for a noncausative, unlinked mutation may also be homozygous for a causative mutation. Usually, this occurs when the number of homozygotes for the noncausative mutation is small. The driven by algorithm omits all instances of shared zygosity for both mutations and recomputes p-values testing departure from the null hypothesis in recessive, additive, and dominant models of transmission, and determines which mutation is the more robust causation candidate. This mutation is assigned “driver” status. Based on driver status together with other factors (e.g., which mutation is the most damaging, which mutation is the most essential for survival to weaning age, and which mutation has evidence of other alleles with a similar phenotype), CE may be able to identify the causative mutation out of a set of colocalizing mutations, giving it a markedly superior CE score.

Finally, an allelic series probed with a phenotypic screen provides an important clue to causation and is considered in CE assessments. If multiple alleles of the same gene are associated with the same phenotype, it is a strong indication that a mutation in this gene caused the observed phenotype. Superpedigrees—composites of multiple pedigrees assayed in the same screen—are of three types. Gene superpedigrees pool different than identical alleles of a given gene, subjected to the same screen. Position superpedigrees pool identical alleles only. Identical alleles may result from: 1) chance mutation of the same nucleotide, 2) transmission of a single mutation to multiple G1 descendants of a single G0 mouse, and 3) a background mutation present in mutagenized stock and shared by multiple G0 mice. Selective gene superpedigrees incorporate only alleles associated with p-values<0.05 with a common direction of effect in a given phenotypic screen, and thus give an intentionally biased view of mutation effects. Because many (but not all) ENU-induced mutations are functionally hypomorphic, a selective gene superpedigree for a set of mutations in a particular gene may strongly implicate that gene in the phenotype probed by the screen in question. The number of pedigrees (and alleles) tested is also important; for very large genes, hundreds of alleles may have been tested, and the finding that two or three alleles score in a particular screen may be due to chance alone. The CE system takes account of this in computing the probability of causation.

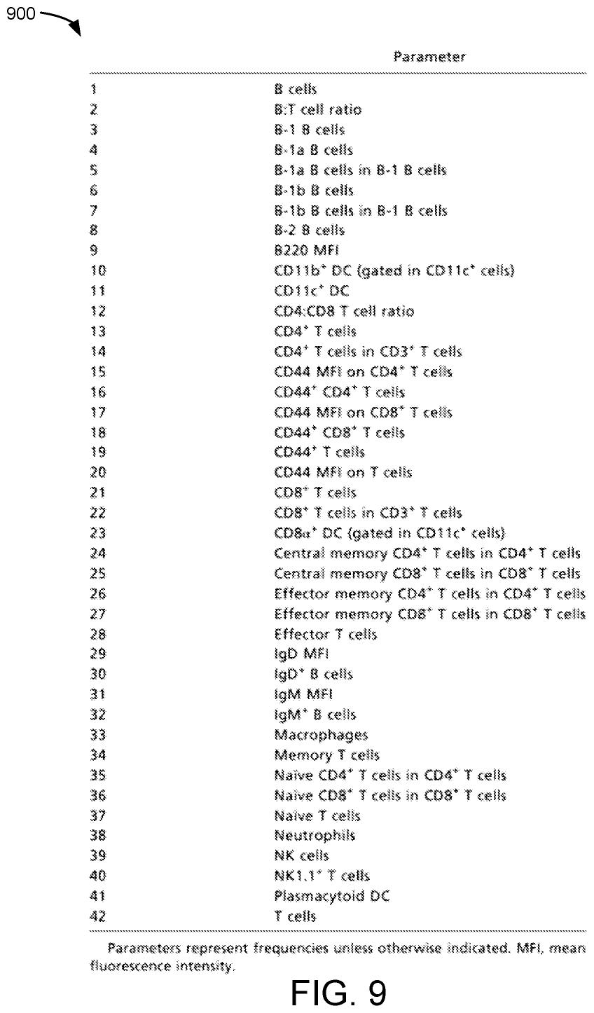

FIG. 9 is a table 900 showing flow cytometry screening parameters, in accordance with certain aspects of the present inventive concept. The flow cytometry screens survey 42 parameters of peripheral blood cells, measuring the frequencies of various immune cell populations and expression levels of several cell surface markers, as shown. Of 7,109,669 mutation-phenotype associations tested by AMM in the flow cytometry screens, 87,795 passed the default initial filters, permitting analysis by CE. These putative mutation-phenotype associations emanated from 39,685 mutations in 14,809 genes, resident in 142,653 G3 mice from 3,987 pedigrees. Restriction to good or excellent candidates reduced the number of mutation-phenotype associations to 7,676, emanating from 2,336 mutations in 1,279 genes, resident in 1,634 pedigrees.

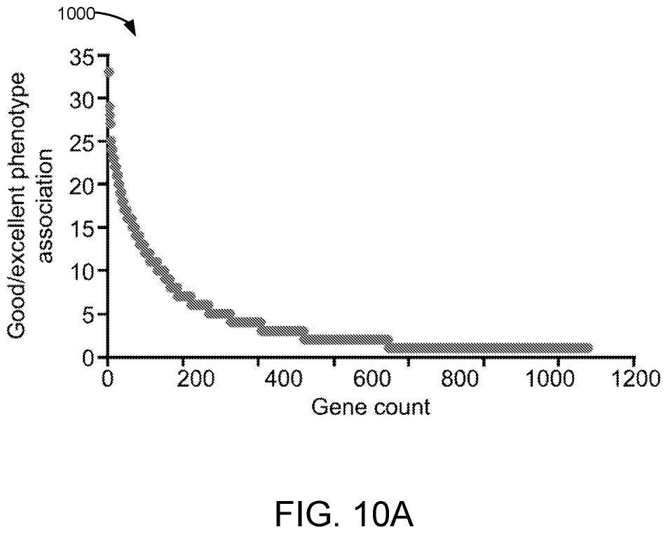

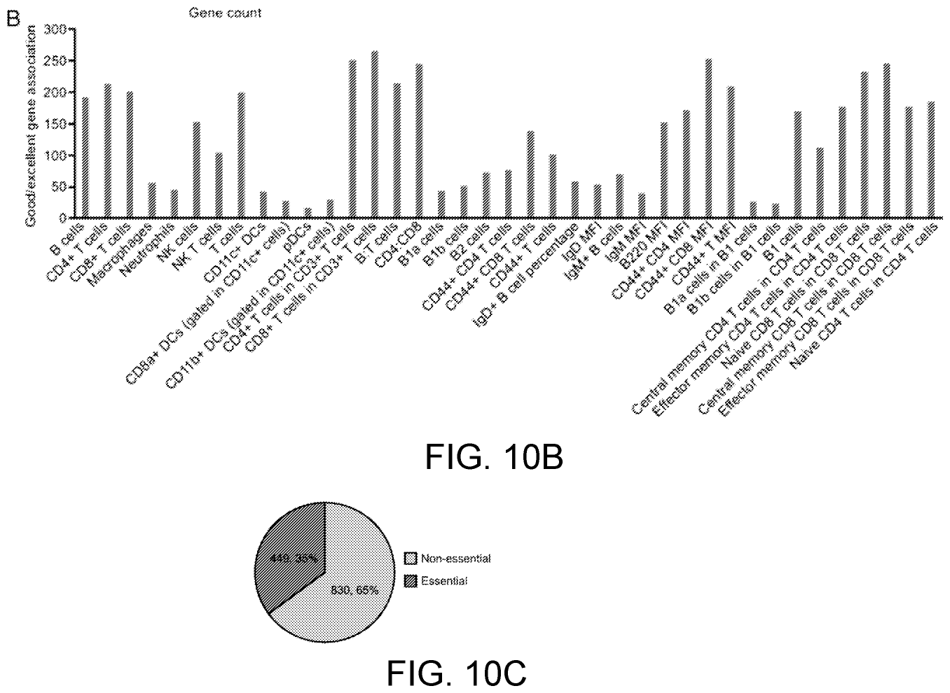

FIGS. 10A, 10B, and 10C illustrate characteristics of gene-phenotype associations for genes with at least one good/excellent mutation-phenotype association, in accordance with certain aspects of the present inventive concept. FIG. 10A is a graph 1000 showing the number of good/excellent phenotype associations plotted versus gene count. FIG. 10B shows the number of good/excellent gene associations plotted versus flow cytometry parameter. FIG. 10C shows the number and percentage of essential and non-essential genes.

Various observations concerning gene-phenotype associations may be made. First, mutations in the majority (872 genes, 68.2%) of the 1,279 genes may result in three or fewer good/excellent phenotype associations, with 533 genes (41.7%) having a single good/excellent phenotype association, as shown in FIG. 10A. In contrast, only 30 genes (2.3%) may have at least 20 good/excellent phenotype associations, and among them, 26 are well-known immune regulatory genes. Second, the number of good/excellent gene associations may vary widely depending on the affected cell type, with B cell and T cell phenotypes associated with the most genes and conventional and plasmacytoid DC phenotypes associated with very few genes shown in FIG. 10B. Finally, 449 genes (35.1%) known or predicted to be essential for viability (E-score>0.55 in this case) may be associated with at least one flow cytometry phenotype, indicating that numerous developmentally important genes likely also have postnatal functions in leukocytes, as shown in FIG. 10C.

A total of 1,354 mutations in 667 genes rated good/excellent by CE and suspected or proven causative of flow cytometry phenotypes may be given allele names and annotated as phenotypic mutations in the Mutagenetix database, irrespective of present candidate status. While named alleles are likely causative, it is uncertain that unnamed alleles are not also causative; indeed, 27% of named alleles had AVP≤0.5. Some of the unnamed alleles are designated as “linked to” or “driven by” another mutation in the same pedigree. This may indicate that they are not causative, but does not always guarantee it, and in some cases two named alleles are linked, suggesting that both mutations may be declared to be causative (e.g., even though they may cosegregate). Evidence for such dual causation may be adduced by CRISPR/Cas9 targeting.

Highly represented gene ontology (G0) annotations associated with the 667 genes with named alleles may be identified. The biological process annotations may be most highly enriched for terms related to immune system processes (211 genes, P=9.82e-42), lymphocyte activation (113 genes, P=5.21e-39), immune system development (117 genes, P=9.73e-36), and other immune development/regulatory processes, which is consistent with manual evaluation identifying 281 (42.1%) of the 667 genes as previously known immune regulators. By manual evaluation, 386 genes represented “new” immunologically important genes, each used for a normal flow cytometry profile. For many of these genes, mutant alleles may not be previously available in mice and no primary immunological or other phenotypic data are available. This may be due in part to known or predicted lethality caused by null alleles of 146 of these 386 genes (E-score>0.5). Enriched G0 terms associated with the 386 new immunologically important genes are dominated by metabolic process terms, including cellular metabolic process (232 genes, P=3.73e-12), organic substance metabolic process (240 genes, P=8.50e-12), cellular macromolecule metabolic process (178 genes, P=3.59e-7), and protein metabolic process (127 genes, P=0.000264). The 386 genes may be assigned to a defined set of broad G0 annotations for biological processes without regard for enrichment. Based on its granular G0 annotations, each gene may be assigned to any of 70 parent G0 terms to which it was related. Notably, 31 of the 386 genes may be associated with the term “immune system process,” based upon genetic interactions, an immune system association of an ancestral gene, sequence orthology to another gene associated with immune system process, or association of the orthologous human gene with an immune system process. In addition, 300 of the 386 genes were detected by RNA-sequencing with medium (11 to 1,000 transcripts per million) or high (>1,000 transcripts per million) expression in the spleen and/or thymus.

At present, a total of 603 genes implicated in flow cytometry phenotypes show a GVP>0.5, 332 genes show a GVP>0.8, and 222 genes show a GVP>0.95. 121 (55%) of genes with GVP>0.95, from which flow cytometry phenotypes are nearly certain to emanate, are known to affect flow cytometry measurements and 101 (45%) are novel.

The CE system allows rapid examination of mutations and genes strongly predicted to affect (or not to affect) phenotypes of interest measured in forward genetic screening. In general, CE provides advantages to human researchers in evaluating mutation-phenotype associations because of its ability to integrate parameters not intuitively favorable or detrimental with respect to linkage analysis, and because it can perform this evaluation more rapidly on a large scale. Using the numerical CE score and categorical assessment given by the CE system, it is simple to rank mutations into priority lists for further in-depth study. In addition, causative mutations can frequently be discerned among several colocalizing mutations. As millions of coding/splicing mutations are introduced into the mouse genome pedigree by pedigree, more extensive allelic series may result, and nearly all genes in which causative loss-of-function mutations can exist may be identified with high confidence.

Beyond its use as a tool for rapid identification of the mutations responsible for ENU-induced phenotypes, the CE system is useful to mouse geneticists studying complex traits (e.g., the Collaborative Cross). Meiotic mapping may confine phenotypes to a relatively large genomic interval, within which many candidate genes with mutational differences exist. If the phenotype is immunologic, knowledge of all genes from which flow cytometric phenotypes emanate is an important starting point for studies of causation, wherein these genes can be targeted.

CE also has value to clinical geneticists seeking to identify the causes of human disease. For patients with immunopathology and flow cytometric anomalies—but no mutation in a “classic” causative gene—other gene variants may be evaluated using CE. Mouse gene symbols corresponding to all loci mutated in the patient (e.g., identified by whole-genome or whole-exome sequencing) may be entered into CE and searched as a batch. Those found to cause a flow cytometric abnormality in the mouse evocative of that in the patient may be considered prime candidates. If the genetic mapping has been performed in a human family and a particular chromosomal region has been identified, identifying a candidate gene can be made with even higher confidence using CE, which also accepts human chromosome coordinates as search input. By using CE in conjunction with analyses of large human genome/phenotype datasets, CE may also facilitate and accelerate the identification of causal variants within disease-associated loci found by genome-wide association studies (GWAS). CE could be queried for relevant mutation-phenotype associations for each candidate gene within a locus identified by GWAS; a mouse gene variant associated with a phenotype similar to the human phenotype under study would suggest causality. Moreover, in most cases, a mutant mouse can be ordered immediately, providing a model of the human disease for laboratory study. Because most mutations cause loss-of-function (e.g., rather than gain-of-function or new functions), and the majority of mouse genes have human orthologs or homologs, many such cases might quickly be solved. Thus, CE is a powerful resource that addresses the question of “missing heritability” associated with immune abnormalities, and as noted for the 386 new genes, genes that regulate or mediate cellular metabolic processes may be prime candidates for consideration.

Mutation-phenotype associations representative of genes with one or more variant alleles and flow cytometric parameters of peripheral blood leukocytes may be evaluated. Flow cytometric analyses allow detecting and measuring immune cell populations with specific functional correlations and provide insight into the developmental stages cells traverse. Abnormal flow cytometry patterns are often associated with immune dysfunction, and many immunodeficiency and autoimmune phenotypes may be initially detected not by functional screens per se, but by analyzing the peripheral blood with flow cytometry. Human disease states, exhibiting similar or identical flow cytometry phenotypes, attest to the clinical relevance of many mouse flow cytometry abnormalities. To date, about 5% genome saturation has been achieved in screening 42 flow cytometry parameters, from which 1,004 genes with good/excellent phenotype associations not previously associated with immune function (from G0 analysis of the 1,279 genes, which found that 275 were associated with “immune system process”) are identified. Thus, even with a false-discovery rate up to 20%, about 456 more new immunologically important genes remain to be found.

In broadly surveying all 1,279 genes with at least one good/excellent phenotype association, a far greater percentage of genes may be observed having one, two, or three good/excellent phenotype associations (68.2%) compared to the percentage with many (≥20) good/excellent phenotype associations (2.3%). These findings suggest that the majority of genes affecting immune cell populations in the blood carry out cell type- or phenotype-specific functions. The hypothesis that identical or similar combinations of phenotypes affected by two or more genes can indicate the functioning of those genes in a common molecular pathway is investigated. Good/excellent gene associations may not affect cell populations with equal frequency despite uniform phenotypic testing across all screens. For example, T cells may have 4.8-fold more gene associations than conventional dendritic cell (DC), 12.5-fold more than plasmacytoid DC, and 4.4-fold more than neutrophils. While a trivial explanation is that significant phenotypic differences are detected less often for rarer blood cell populations, another possibility reflecting the biology of cells is that T cells are intrinsically less tolerant of genetic variation than conventional DC, plasmacytoid DC, or neutrophils, at least with respect to the numbers of these cells represented in the peripheral blood. An understanding of individual protein function and the pathways they regulate is important to gain insight into these issues.

The vast majority of mice phenotyped by flow cytometry are also phenotyped in other screens, among them screens measuring responses to immunization, innate immune responses, body weight, blood pressure, heart rate, dextran sodium sulfate (DSS) sensitivity, circadian rhythms, and motor coordination. Data from screens for skeletal phenotypes detected by DEXA scanning are currently publicly accessible. In the future, the data from other screens may be released for public users of CE to interpret a wide range of phenotypic consequences that emanate from each mutation. All biomedically relevant phenotypic screens may ultimately enlighten the study of human phenotype and help to distinguish mechanisms of phenotypes caused by certain alleles, as many mutations score in disparate screens (for example, immune function and body weight, or immune function and neurobehavioral function).

The following provide further details regarding materials and method which may be used for identifying associations. Eight to 10-wk-old C57BL/6J males may be mutagenized with ENU. As described, mutagenized G0 males may be bred to C57BL/6J females, and the resulting G1 males may be crossed to C57BL/6J females to produce G2 mice. G2 females may be back-crossed to their G1 sires to yield G3 mice, which are screened for phenotypes. Whole-exome sequencing and mapping may be performed.

To generate mice carrying CRISPR/Cas9-targeted mutations, female C57BL/6J mice may be superovulated by injection with 6.5 U pregnant mare serum gonadotropin (PMSG; Millipore), then 6.5 U human chorionic gonadotropin (hCG; Sigma-Aldrich) 48 h later. The superovulated mice were subsequently mated with C57BL/6J male mice overnight. The following day, fertilized eggs may be collected from the oviducts and in vitro transcribed Cas9 mRNA (50 ng/μL) and small base-pairing guide RNA (50 ng/μL) were injected into the cytoplasm or pronucleus of the embryos. The injected embryos were cultured in M16 medium (Sigma-Aldrich) at 37° C. and 5% CO2. For the production of mutant mice, two-cell stage embryos may be transferred into the ampulla of the oviduct (10 to 20 embryos per oviduct) of pseudopregnant Hsd:ICR (CD-1) (Harlan Laboratories) females.

Peripheral blood may be collected from G3 mice greater than 6 weeks old by cheek bleeding. Red blood cells (RBCs) may be lysed with a hypotonic buffer (eBioscience). Samples may be washed with FACS staining buffer (phosphate buffered saline (PBS) with 1% [weight/volume] bulked-segregant analysis (BSA)) one time and then centrifuged at 500×g for 5 min. The RBC-depleted samples may be stained for 1 hour at 4° C., in 100 μL of a 1:200 mixture of fluorescence-conjugated antibodies to 15 cell surface markers encompassing the major immune lineages B220 (BD, clone RA3-6B2), CD19 (BD, clone 1D3), IgM (BD, clone R6-60.2), IgD (BioLegend, clone 11-26c.2a), CD3ε (BD, clone 145-2C11), CD4 (BD, clone RM4-5), CD8α (BioLegend, clone 53-6.7), CD11b (BioLegend, clone M1/70), CD11c (BD, clone HL3), F4/80 (Tonbo, clone BM8.1), CD44 (BD, clone 1M7), CD62L (Tonbo, clone MEL-14), CD5 (BD, clone 53-7.3), CD43 (BD, clone S7), NK 1.1 (BioLegend, clone OK136), and 1:200 Fc block (Tonbo, clone 2.4G2). Flow cytometry data may be collected on a cell analyzer (e.g., BD LSR Fortessa) and the proportions of immune cell populations in each G3 mouse may be analyzed with software for analyzing cytometry data. The resulting phenotypic data may be uploaded to a server (e.g., Mutagenetix) for automated mapping of causative alleles.

AMM may be performed as described herein. For example, genotypes at all mutation sites present in the exomes of G3 mice may be determined prior to phenotypic screening. Tail DNA from G1 males may be subjected to whole-exome sequencing using a sequencing instrument (e.g., Illumina HiSEq. 2500). G2 and G3 mice may then be genotyped at the identified mutation sites (e.g., using an Ion PGM (Life Technologies)). Following the phenotypic screening, linkage analysis using recessive, additive, and dominant models of inheritance may be performed for every mutation in the pedigree using the program Linkage Analyzer; phenotypic data scatter plots and Manhattan plots may be displayed using the program Linkage Explorer. The p-values of association between genotype and phenotype may be calculated using a likelihood ratio test from a generalized linear model or generalized linear mixed-effect model and Bonferroni correction applied.

In some aspects, the CE prediction model may be built using a random forest algorithm (e.g., implemented in an R classification and regression training (caret) package. Linkage data obtained through screening may be released in phases according to phenotype.

The damage score is an ensemble score that uses a logistic regression model to integrate independent prediction scores (e.g., 38 scores). Thirty-seven prediction scores may be retrieved from a database (e.g., the human dbNSFP), and includes scores from the following algorithms: SIFT, SIFT4G, Polyphen2-HDIV, Polyphen2-HVAR, LRT, MutationTaster2, MutationAssessor, FATHMM, MetaSVM, MetaLR, CADD, CADD_hg19, VEST4, PROVEAN, FATHMM-MKL coding, FATHMM-XF coding, fitCons (four scores), LINSIGHT, DANN, GenoCanyon, Eigen, Eigen-PC, M-CAP, REVEL, MutPred, MVP, MPC, PrimateAl, GEOGEN2, BayesDel_addAF, Bayes Del_noAF, ClinPred, LIST-S2, and ALOFT. The dataset may use the ranked scores of each algorithm transformed by dbNSFP. The 38th prediction score is the probability of protein damage to phenovariance caused by mouse mutations, calculated as described herein. Among the 38 prediction scores, the score from MutPred is important, along with the probability of protein damage to phenovariance caused by mouse mutations, and phastCons100way_vertebrate (a conservation score). Damage Score may be used as a quantitative prediction score to measure the likelihood of a mouse mutation being deleterious.

If a mouse missense mutation is the same as a human mutation (e.g., both nucleotide and amino acid changes), then the mutation effect in human and mouse may be similar. Therefore, human scores may be used to predict the likelihood of damage in mice. A set of mouse ENU mutations with class tags (e.g., known damaging or neutral) may be retrieved from the Mutagenetix database. The known mutation class tags come from four sources: 1) Physically isolated mutations (of linkage with all other coding/splicing mutations in the pedigree) that fall within essential genes yet can be transmitted from heterozygous G2 females and their heterozygous G1 sire to homozygous G3 mice at a ratio that does not significantly depart from Mendelian expectation, are considered neutral. 2) conversely, isolated mutations in important genes that are not transmitted to homozygosity, to the extent that homozygotes are observed at frequencies significantly beneath the expected Mendelian ratio, are considered damaging. 3) mutations that cause qualitative (usually visible) phenotypes are considered damaging. 4) mutations that have been verified to be significant in phenotypic screening of CRISPR replacement alleles are also considered to be damaging.

The mutations tagged as damaging or neutral may be lifted over from mouse genome to human genome (translated to the equivalent amino acid) and kept for mutations that lead to the same nucleotide and amino acid changes in both genomes. About 4% of mouse mutations may not be mapped to the corresponding human mutations using a lift-over tool for converting genome coordinates and annotation files between assemblies. They may not be included in the final dataset for model training and testing. A point-biserial correlation may be used to estimate the relationship between the mouse mutations tagged damaging or neutral with the most important human mutation prediction score. The correlation coefficient may be 0.525, with 95% CI: 0.50 to 0.55. Then corresponding human mutations may be searched in the dbNSFP database to obtain scores for all available prediction methods. The retrieved scores, combined with the probability of phenotypically detectable damage by the mutations in mice, may be integrated with the input dataset and used to train and optimize a logistic regression model using the train function of the R caret package with 10-fold cross-validation. This process may be repeated multiple times (e.g., three times). The scaling of the data may be performed by the preprocess function. The constructed model (classifier) may be then used to compute the score of a set of mutations with unknown class membership. The dataset used for prediction may be created in the same way as dataset used for modeling. The score predicted by the model represents the probability of a mutation being in the damaging class. The higher the score, the more likely to be deleterious the mutation.

An input dataset may contain mouse mutations (e.g., 3,334 mutations), of which a portion (e.g., 1,088) are deleterious and a portion (e.g., 2,246) are neutral. In order to evaluate the performance of the constructed model in predicting the membership of the new mutation category, the input dataset may be randomly divided into two sets: one set consisting of mutations (e.g., 2,668 mutations, 80% of original dataset, 871 deleterious mutations and 1,797 neutral mutations) may be used to train and validate the logistic regression model, and a second set of the remaining 666 mutations may be used to test the performance of the established model. The 80/20 splits for training and testing may be conducted multiple (e.g., 10) times randomly. The ROC curve may yield an AUC close to the average AUC value of 0.853±0.014.

E-score may be used to estimate the likelihood of lethality in mice when the gene is knocked out. Essential and non-essential genes in mice can be distinguished by various independent features of genes. The logistic regression method is used to fit the features of known essential and non-essential genes in mice to obtain a trained model for predicting the unknown essentiality of genes.

The model uses the following gene features: 1) from the online gene essentiality (OGEE) database: gene conservation, connectivity in protein-protein interaction network, expression stage during development, evolutionary age, G0 terms, copy number of genes, and length of gene product, where the features are associated with gene essentiality of many species, including mice, 2) the essentiality of human orthologous genes: the genes for cell proliferation and viability in tested cell lines may be defined as important genes under specific conditions, frequency of being important in tested human cell lines may be used as a feature in the model, 3) probability of loss-of-function intolerance (pLI) score from the Exome Aggregation Consortium (ExAC): the closer the score is to 1, the more likely the gene is essential to human survival, 4) minimum p-values for an ENU-targeted mouse gene obtained from the lethal model by the Linkage Analyzer algorithm.

The phenotypic description of the 8,032 genes in mouse genome informatics (MGI), which may be knocked out in mice, may be carefully reviewed, and a set of genes designated as “essential” or “non-essential” may be manually curated according to the following criterion: 1) If the homozygous knockout allele is explicitly described as causing embryonic lethality, neonatal lethality, prenatal lethality, perinatal lethality, or preweaning lethality, the gene may be considered to be required to survive before weaning and may be classified as an essential gene, an E-score of 1 may be assigned to the gene, 2) If homozygous knockout alleles are compatible with viability, normal growth, no obvious phenotype, or some phenotype, but not apparent effect on viability, then it may be classified as a non-essential gene, an E-score of 0 may be assigned to the gene. In addition, an E-score of 1 may be assigned to those genes verified in our CRISPR knockout experiments as causing significant lethality before weaning; an E-score of 0 may be assigned to genes verified in our CRISPR knockout experiments as resulting in normal mendelian ratios in crosses of heterozygous mutants.

A set of 7,009 genes, in which 2,587 may be labeled as essential genes and 4,422 as non-essential genes, may be integrated with the described gene features. The resulting dataset may be used to train and optimize a logistic regression model using the train function of the R caret package with 10-fold cross-validation, which may be repeated multiple times (e.g., three times). The scaling of the data may be performed by the preProcess function. The function preProcess estimates the required parameters for each operation. The constructed model may be then used to predict the essentiality of remaining mouse genes. The predicted score is between 0 and 1. The closer the score is to 1, the more likely the gene is essential.

To assess the performance of constructed model in predicting unknown essentiality of genes, the dataset used to construct the model may be randomly divided into two sets: one set including 5,608 genes (80% of original dataset, 3,538 non-essential genes and 2,070 essential genes) may be used to train and validate the logistic regression model, and the remaining 1,401 genes may be used to test the performance of the established model in the training dataset. The 80/20 splits for training and testing may be conducted multiple times (e.g., 10 times) randomly; the ROC curve yielding an AUC close to the average AUC value of 0.891±0.0087.

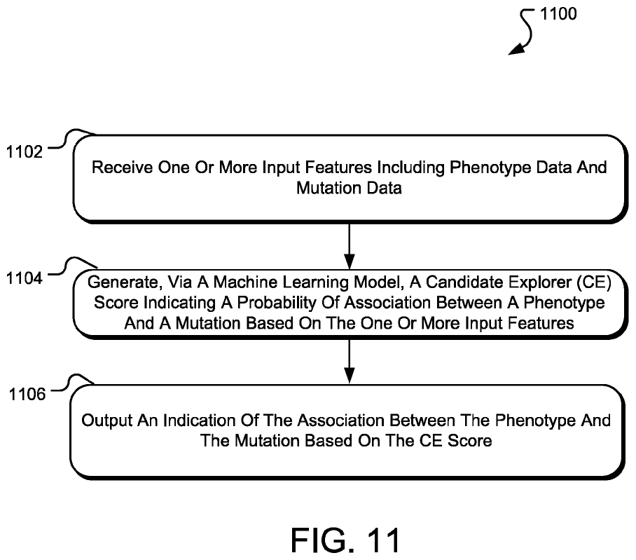

FIG. 11 is a flow diagram illustrating example operations 1100 for mutation processing, in accordance with certain aspects of the present inventive concept. The operations 1100 may be performed, for example, by a CE system such as the processor 103 and the memory 115.

At block 1102, the CE system may receive one or more input features including phenotype data and mutation data. At block 1104, the CE system may generate, via a machine learning model, a CE score indicating a probability of association between a phenotype and a mutation based on the one or more input features

In some aspects, the mutation data may also include a damage score indicating a likelihood that a protein associated with the mutation is functionally impaired. For example, the CE system may generate the damage score via another machine learning model trained using known deleterious and neutral mutations.

In some aspects, the one or more input features may also include an essentiality score indicating a likelihood of lethality prior to weaning age in mice homozygous for a robust knockout allele of a gene associated with the mutation. The CE system may generate the essentiality score via another machine learning model trained using genes that are known to be non-essential for survival and genes that are known to be essential for survival. In some aspects, the one or more input features also include a feature associated with an algorithmic score indicating a likelihood that the mutation is causative.

In some aspects, the one or more input features also include linkage data generated using automated meiotic mapping (AMM) (e.g., as performed by a linkage analyzer algorithm or program). In certain aspects, when two or more mutations are cosegregated, the CE system may determine which of the two or more mutations is a more robust causation candidate for the phenotype by omitting instances of shared zygosity for the two or more mutations. The CE score may be generated at block 1104 based on the determination.

In some aspects, the one or more input features may include, one or any combination of the following: number of phenotypes with an algorithmic score for the mutation that meets a threshold, the algorithmic score indicating a likelihood that the mutation is causative; average number of automated meiotic mapping (AMM) operations resulting in a p value that meets a threshold for each allele of a gene associated with the mutation; the algorithmic score for the mutation or phenotype; number of AMM operations resulting in a p-value that meets a threshold for the gene associated with the mutation; damage score for the mutation, the damage score indicating a likelihood that a protein associated with the mutation is functionally impaired; number of pedigrees in a superpedigree associated with the gene and whether a p-value resultant from AMM operation for the superpedigree meets a threshold; number of phenotypes with a p-value for the superpedigree that meets a threshold; number of pedigrees contributing to a p-value for the superpedigree that meets a threshold; number of pedigrees in the superpedigree; percentage of fluorescence activated cell sorting (FACS) screens with a p-value that meets a threshold for the mutation; a minimum of the p-value from the AMM operations; percentage of variant allele (VAR) mice with screen results that overlap with those of B6 mice; whether AMM operations results for the superpedigree meets a threshold for null and missense alleles; whether AMM operations results for the superpedigree meets a threshold for null alleles; percentage of VAR mice with screen results that overlap with those of reference allele (REF) mice; difference between results of AMM operations for heterozygous (HET) and VAR mice; number of female REF mice used for the AMM operations; percentage of body weight screens with a p-value that meets a threshold for the mutation; number of female HET mice used for the AMM operations; or difference between results of AMM operations for REF and VAR mice.

At block 1106, the processing system may output the CE score. For example, in some aspects, the processing system may generate a candidate status (e.g., excellent, good, potential, or not good candidate) for the association between the phenotype and the mutation based on the CE score and an algorithmic score indicating a likelihood that the mutation is causative.

These and various other arrangements will be described more fully herein. As will be appreciated by one of skill in the art upon reading the following disclosure, various aspects described herein can be a method, a computer system, or a computer program product. Accordingly, those aspects can take the form of an entirely hardware implementation, an entirely software implementation, or at least one implementation combining software and hardware aspects. Furthermore, such aspects can take the form of a computer program product stored by one or more computer-readable storage media (e.g., non-transitory computer-readable medium) having computer-readable program code, or instructions, included in or on the storage media. Any suitable computer-readable storage media can be utilized, including hard disks, CD-ROMs, optical storage devices, magnetic storage devices, and/or any combination thereof. In addition, various signals representing data or events as described herein can be transferred between a source and a destination in the form of electromagnetic waves traveling through signal-conducting media such as metal wires, optical fibers, and/or wireless transmission media (e.g., air and/or space).

Implementations of the present inventive concept include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

While specific implementations are discussed, it should be understood that this is done for illustration purposes only. A person skilled in the relevant art will recognize that other components and configurations may be used without parting from the spirit and scope of the present inventive concept. Thus, the following description and drawings are illustrative and are not to be construed as limiting. Numerous specific details are described to provide a thorough understanding of the present inventive concept. However, in certain instances, well-known or conventional details are not described in order to avoid obscuring the description. References to one or an implementation in the present inventive concept can be references to the same implementation or any implementation; and such references mean at least one of the implementations.

Reference to “one implementation” or “an implementation” means that a particular feature, structure, or characteristic described in connection with the implementation is included in at least one implementation of the present inventive concept. The appearances of the phrase “in one implementation” in various places in the specification are not necessarily all referring to the same implementation, nor are separate or alternative implementations mutually exclusive of other implementations. Moreover, various features are described which may be exhibited by some implementations and not by others.

The terms used in this specification generally have their ordinary meanings in the art, within the context of the present inventive concept, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the present inventive concept or of any example term. Likewise, the present inventive concept is not limited to various implementations given in this specification.

Without intent to limit the scope of the present inventive concept, examples of instruments, apparatus, methods and their related results according to the implementations of the present inventive concept are given below. Note that titles or subtitles may be used in the examples for convenience of a reader, which in no way should limit the scope of the present inventive concept. Unless otherwise defined, technical and scientific terms used herein have the meaning as commonly understood by one of ordinary skill in the art to which the present inventive concept pertains. In the case of conflict, the present document, including definitions will control.

Additional features and advantages of the present inventive concept will be set forth in the description which follows, and in part will be obvious from the description, or can be learned by practice of the herein disclosed principles. The features and advantages of the present inventive concept can be realized and obtained by means of the instruments and combinations particularly pointed out in the appended claims. These and other features of the present inventive concept will become more fully apparent from the following description and appended claims or can be learned by the practice of the principles set forth herein.

What is claimed is:

1. A method for mutation processing comprising:
receiving one or more input features including phenotype data and mutation data;
generating, via a machine learning model, a candidate explorer (CE) score indicating a probability of association between a phenotype and a mutation based on the one or more input features; and
outputting an indication of the association between the phenotype and the mutation based on the CE score.

2. The method of claim 1, wherein the indication of the association includes a candidate status for the association based on the CE score and an algorithmic score indicating a likelihood that the mutation is causative.

3. The method of claim 1, wherein the mutation data includes a damage score indicating a likelihood that a protein associated with the mutation is functionally impaired.

4. The method of claim 3, further comprising:
generating the damage score via another machine learning model trained using known deleterious and neutral mutations.

5. The method of claim 1, wherein the one or more input features further includes an essentiality score indicating a likelihood of lethality prior to weaning age in mice homozygous for a robust knockout allele of a gene associated with the mutation.

6. The method of claim 5, further comprising:
generating the essentiality score via another machine learning model trained using genes that are known to be non-essential for survival and genes that are known to be essential for survival.

7. The method of claim 1, wherein the one or more input features further includes a feature associated with an algorithmic score indicating a likelihood that the mutation is causative.

8. The method of claim 1, wherein the one or more input features further include linkage data generated using automated meiotic mapping (AMM).

9. The method of claim 1, further comprising:
when two or more mutations are cosegregated, determining which of the two or more mutations is a more robust causation candidate for the phenotype by omitting instances of shared zygosity for the two or more mutations, wherein the CE score is generated based on the determination.

10. The method of claim 1, wherein the one or more input features includes at least one of:
number of phenotypes with an algorithmic score for the mutation that meets a threshold, the algorithmic score indicating a likelihood that the mutation is causative;
average number of AMM operations resulting in a p-value that meets a threshold for each allele of a gene associated with the mutation;
the algorithmic score for the mutation or phenotype;
a number of AMM operations resulting in a p-value that meets a threshold for the gene associated with the mutation;
a damage score for the mutation, the damage score indicating a likelihood that a protein associated with the mutation is functionally impaired;
a number of pedigrees in a superpedigree associated with the gene and whether a p-value resultant from AMM operation for the superpedigree meets a threshold;
a number of phenotypes with a p-value for the superpedigree that meets a threshold;
a number of pedigrees contributing to a p-value for the superpedigree that meets a threshold;
a number of pedigrees in the superpedigree;
a percentage of fluorescence activated cell sorting (FACS) screens with a p-value that meets a threshold for the mutation;
a minimum of the p-value from the AMM operations;
a percentage of variant allele (VAR) mice with screen results that overlap with those of B6 mice;
whether AMM operations results for the superpedigree meets a threshold for null and missense alleles;
whether AMM operations results for the superpedigree meets a threshold for null alleles;
a percentage of VAR mice with screen results that overlap with those of reference allele (REF) mice;
a difference between results of AMM operations for heterozygous (HET) and VAR mice;
a number of female REF mice used for the AMM operations;
a percentage of body weight screens with a p-value that meets a threshold for the mutation;
a number of female HET mice used for the AMM operations; or
a difference between results of AMM operations for REF and VAR mice.

11. An apparatus for mutation processing comprising:
a memory; and
one or more processors coupled to the memory and configured to:
receive one or more input features including phenotype data and mutation data;
generate, via a machine learning model, a candidate explorer (CE) score indicating a probability of association between a phenotype and a mutation based on the one or more input features; and
output an indication of the association between the phenotype and the mutation based on the CE score.

12. The apparatus of claim 11, wherein the indication of the association includes a candidate status for the association based on the CE score and an algorithmic score indicating a likelihood that the mutation is causative.

13. The apparatus of claim 11, wherein the mutation data includes a damage score indicating a likelihood that a protein associated with the mutation is functionally impaired.

14. The apparatus of claim 13, wherein the one or more processors are further configured to generate the damage score via another machine learning model trained using known deleterious and neutral mutations.

15. The apparatus of claim 11, wherein the one or more input features further includes an essentiality score indicating a likelihood of lethality prior to weaning age in mice homozygous for a robust knockout allele of a gene associated with the mutation.

16. The apparatus of claim 15, wherein the one or more processors are further configured to generate the essentiality score via another machine learning model trained using genes that are known to be non-essential for survival and genes that are known to be essential for survival.

17. The apparatus of claim 11, wherein the one or more input features further includes a feature associated with an algorithmic score indicating a likelihood that the mutation is causative.

18. The apparatus of claim 11, wherein the one or more input features further include linkage data generated using automated meiotic mapping (AMM).

19. The apparatus of claim 11, wherein, when two or more mutations are cosegregated, the one or more processors are further configured to determine which of the two or more mutations is a more robust causation candidate for the phenotype by omitting instances of shared zygosity for the two or more mutations, wherein the one or more processors are configured to generate the CE score based on the determination.

20. A non-transitory, computer-readable medium storing instructions that, when executed by one or more processors, cause the one or more processors to:
receive one or more input features including phenotype data and mutation data;
generate, via a machine learning model, a candidate explorer (CE) score indicating a probability of association between a phenotype and a mutation based on the one or more input features; and
output an indication of the association between the phenotype and the mutation based on the CE score.
